(19) United States
(12) Patent Application Publication
(10) Pub. No.: US 2019/0321661 A1
(43) Pub. Date: Oct. 24, 2019

(54) FACE MASK FOR REGULATING HEAT AND HUMIDITY

(71) Applicant: Orindi Ventures, LLC, Holland, MI (US)

(72) Inventor: Jordan R. Vanderham, Holland, MI (US)

(73) Assignee: Orindi Ventures, LLC

(21) Appl. No.: 16/388,259

(22) Filed: Apr. 18, 2019

Related U.S. Application Data
(60) Provisional application No. 62/659,311, filed on Apr. 18, 2018.

(51) Int. Cl. A62B 9/00; A61M 16/06; A61M 16/10

(52) U.S. Cl. CPC A62B 9/003; A61M 2205/0205; A61M 16/1045; A61M 16/0605

(57) ABSTRACT

A mask or mask component having air heating and humidity retention properties and a passive speaking membrane. The mask includes a heat exchanger and a plurality of corresponding air channels. In one embodiment, silver plated copper mesh is used as the heat exchanger. During the exhaling process, the heat exchanger captures heat and humidity from the exhaled air of the user wearing the mask. Further, during the inhaling process, the heat exchanger transfers the captured heat and moisture to the air being inhaled by the user. Condensed water is re-humidified into entering air that has passed through the heat exchanger. The passive speaking membrane permits and is tuned to allow and amplify the passage of the user's voice for better communication.

### FIELD OF THE INVENTION

The present specification generally relates to face masks and more particularly, to face masks for regulating heat and humidity and enabling voice communication thorough a passive speaking membrane and a porous heat exchanger.

### BACKGROUND

Inhaling cold air into the respiratory tract can be detrimental to the health of persons suffering from emphysema, asthma, angina and various other ailments. In cold weather, persons suffering from such ailments must either avoid breathing cold air altogether or take precautions to precondition (heat up and humidify) cold air before it is breathed. In addition, persons of good health working and exercising in frigid climates must take precautions against excessive heat and water loss due to the inhalation of frigid and low humidity air.

Persons who work in cold environments need to take precautions to avoid cold fatigue and lung spasms when being in a cold environment for a long duration. Lung spasms occur when the body is unable to naturally regulate the inhaled air to approximately 98.6 degrees Fahrenheit and 100 percent humidity. The transition point between properly regulated air at approximately 98.6 degrees Fahrenheit and 100 percent humidity and incoming air is called the isothermic saturation boundary. When cold low humidified air is present at this boundary it lowers the isothermic saturation boundary towards the lungs and lung spasms can be triggered.

Many breathing masks have been developed for protecting humans from exposure to extreme weather, such as cold. Masks have also been developed to protect humans from a variety of particulate and gaseous matter. However, prior masks have been unsuccessful in supplying sufficiently heated and humidified air to persons having respiratory and heart ailments to enable them to move about and work normally outside in cold weather without experiencing discomfort and pain. Additionally, prior masks have been unsuccessful in supplying sufficiently heated and humidified air to persons who work in a cold environment for a long duration (more than 4 hours with properly allotted breaks).

Typically, this discomfort and pain is experienced by persons having respiratory conditions such as asthma, bronchitis, chronic bronchitis, emphysema or coronary conditions such as angina pectoris, post myocardial infarction, congestive heart failure, coronary heart disease, post coronary bypass and the like. Usually such persons experience sufficient pain and discomfort that they must cease exerting themselves and get into a warm environment and rest. Hence, their activity in cold weather must be severely curtailed and, in some instances, substantially eliminated.

A variety of apparatuses have been employed in the past to overcome the ill effects of breathing cold air. These range from simple scarfs to complex breathing masks and are employed to preheat cold air prior to inhalation. Most of these devices, however, either do not sufficiently preheat and humidify the cold surrounding air before breathing or are complex, cumbersome and prohibitively expensive. These mask apparatuses which attempt to regulate air are an inefficient application of heat exchangers, reservoirs, or phase change materials. In most of these devices, condensation from the user's breath builds up in the textile or plastic and rests on the user's face. This water gets cold or freezes and can cause damage to the user's face like frostbite. Existing masks with heating elements are in efficient and have been inconvenient to use under normal use conditions because large, poorly located, poorly implemented battery power supplies are either separate from the mask and require additional means for transport or are located within the mask breathing space where the battery is exposed to high moisture levels and where a user would be subjected to dangers from battery corrosion or battery fumes.

Another problem experienced with existing masks having heating elements is that a large amount of power is required to directly heat cold surrounding air. Existing masks also lack the capability to speak clearly thought the mask. Accordingly, a mask overcoming the aforementioned disadvantages is desirable.

### SUMMARY OF THE INVENTION

The mask as described with regard to the embodiments herein is designed to both regulate temperature and humidity of breath taken by a user while wearing the mask. The regulation unit is designed to passively increase the temperature and humidity that the user intakes while breathing in and to capture heat and humidity in the user's breath while breathing out. The temperature regulation portion of the mask captures the heat and humidity of the user's breath while breathing out and applies it to the user's breath while breathing in. The temperature regulation unit includes a passageway in which air is directed through a material with a high thermal conductivity.

During operation of the mask by the user, water is condensed on the internal surfaces of the mask. The phase change of the humidity in the user's breath is used to condensate releases more energy into the highly thermal conductive material, as opposed to not condensing the humidity, resulting in higher temperature regulation efficiency and capture of water. The condensed water does not sit and collect in the bottom of the mask, but rather wicks up in the internal surfaces of the mask to increase surface area and evaporation. This increases the user's inhaled humidity. Adding a layer of open cell foam that the air passes through could also increase the user's inhaled humidity. This technique is used in the medical field with humidity moisture exchangers on tracheostomy tubes.

As described herein, the materials used to construct the mask have a high thermal conductivity as well as a high heat capacity or thermal capacity to more efficiently store and release heat quickly. A high surface area throughout promotes heat conduction and convection within the apparatus. The material with a high thermal conductivity, the passageway, and/or other components of the mask may have a surface treatment to keep the mask hygienic. Texturing, silver, or a variation of Quantum Ammonia can be used as a surface treatment. Surface treatment can be done through plating, spraying small particles, soaking, and/or spraying with an electrostatic sprayer.

The mask is also configured to regulate the humidity of the air inhaled by the user. In one embodiment, condensed air and water is pulled and spread across the surface of the mask increasing the surface area and evaporation rate. This condensation and water is pulled to the surface using a vertical and/or horizontal wicking porous matrix and leveraging the surface tension of water. A grained texture on the internal surface of the mask could also encourage condensation movement and placement on more vertical surfaces. Ridges positioned closely adjacent to each other also wick the water across the surface of the mask. In some embodiments, a piezoelectric humidifier or ultrasonic humidifier could be used to atomize the condensation. The re-vaporized water would then be taken into the body when the user breathes in.

These and additional features provided by the various embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 illustrates a partially exploded perspective view of an embodiment of the mask according to one or more embodiments shown and described herein;

FIG. 2 illustrates an assembled perspective view of the mask as shown in FIG. 1;

FIG. 3 illustrates a rear perspective view of the mast shown in FIG. 1;

FIG. 4 illustrates a partially exploded perspective view of an alternative embodiment of the mask as shown and described herein;

FIG. 5 illustrates an assembled perspective view of the mask shown in FIG. 5;

FIG. 6 illustrates a rear perspective view of the mask shown in FIG. 5;

FIG. 7 illustrates a cross-sectional side view of a speaking membrane as used in the mask according to one or more embodiments of the invention;

FIG. 8 illustrates a front view of the speaking membrane as shown in FIG. 7;

FIG. 9 illustrates a user wearing the mask according to one or more embodiments shown and described herein.

FIG. 10 illustrates a partially exploded perspective view according to yet another embodiment of the invention.

FIG. 11 illustrates an assembled perspective view of the mask shown in FIG. 10.

FIG. 12 illustrates a rear perspective view of the mast shown in FIG. 10.

FIG. 13 illustrates is a partially exploded view of the mask in FIG. 10 where the body removed from the mask.

FIG. 14 illustrates a cross-sectional rear perceptive view of the mask shown in FIG. 10.

### DETAILED DESCRIPTION

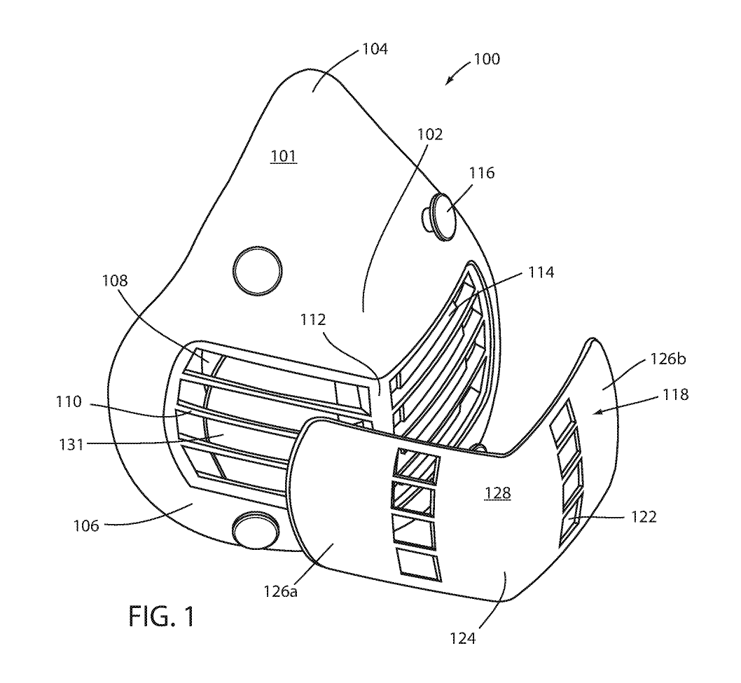

FIG. 1 generally depicts a mask having air heating and humidity retention properties. The mask includes a heat exchanger and a plurality (2 or more) of corresponding air channels. In one embodiment, silver plated copper mesh is used as the heat exchanger. During the exhaling process, the heat exchanger captures heat and humidity from the exhaled air of the user wearing the mask. Further, during the inhaling process, the heat exchanger transfers the captured heat and moisture (humidity retention) to the air being inhaled by the user.

Referring now to FIGS. 1-3, in this first embodiment, a mask 100 is provided. The mask 100 generally includes a body 101 having a central portion 102. An upper portion 104 and a lower portion 106 are provided as reference points. The body 101 is preferably made of a silicone or other suitable material having properties similar to silicone, plastic or equivalent materials. The body 101 is shaped so as to confirm, or at least partially follow, the contour of the user's face. The upper portion is designed to fit over the user's nose while the lower portion rests at the user's chin, below the user's chin, or just below the mouth (depending on the exact design and the user's face size). The mask is configured to cover both the nose and the mouth to assist in temperature and humidity regulation of air exhaled and inhaled from both the nose and the mouth.

Furthermore, a textile garment (as illustrated in FIG. 9) is provided to connect to the grommet hooks 116. The textile garment is configured to wrap around the back of the user's head to hold the mask in place. The textile garment may also be provided to supply additional warmth to the user.

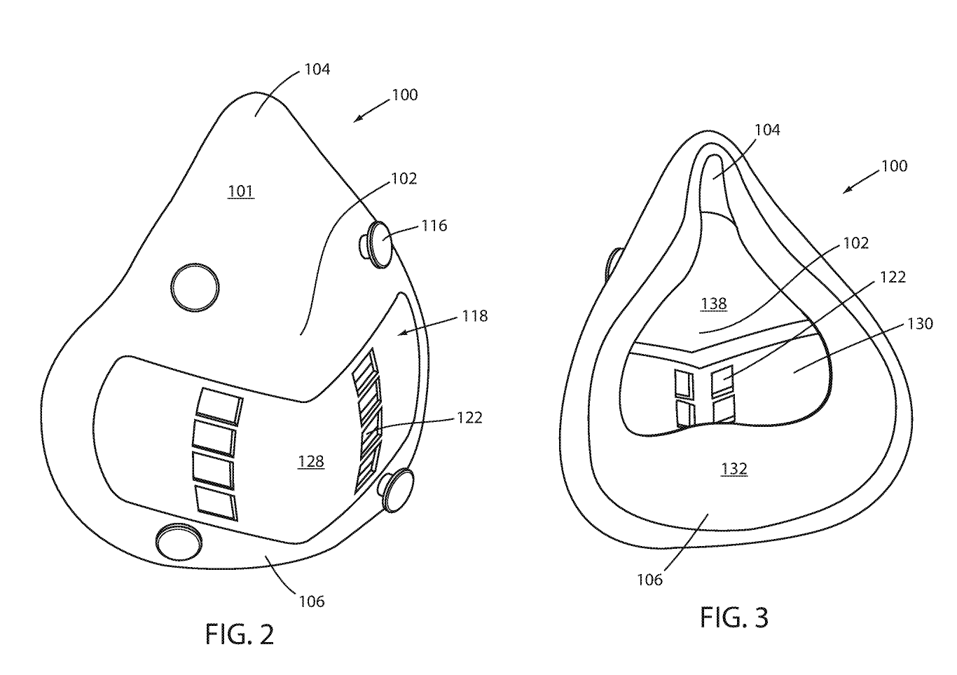

The mask 100 further includes a removeable cover 118 configured to cover the air channels or passageways 114. The cover is 118 is slightly folded or bent at its center 124. As seen in FIG. 2, the outer surface 128 has side portions 126a and 126a where each respective side portion includes a plurality of apertures 122 and 108 to further assist and regulate airflow to the air channels on the body 101 of the mask 100. Although shown in an elongated rectangular configuration those skilled in the art will recognize that other shapes are possible. An area 124 is located between each respective group of apertures 122 has no opening and is used to cover the center portion of the mask 101. As seen in FIG. 3, the rear of the mask 100 includes a surface 132. The surface 132 is configured to also contour the shape of the user's face while the mask is formed and in use. The mask 100 includes an inner surface 138 and 130 which may also include an antimicrobial surface and/or coating e.g. silver or the like.

Those skilled in the art should recognize that the masks 100, 200, and 400 as described herein, are used both for their temperature regulation and humidity regulation properties. To achieve temperature regulation, exhaled air warms and raises the temperature of a copper heat exchanger. Thereafter, inhaled air is warmed up by the heat exchanger. A copper mesh is silver plated to inhibit bacteria growth. To regulate humidity, the heat exchanger is used to condense exhaled humidity. The heat exchanger or duct portion does not become saturated in water, rather, the condensed water wicks across the inner surface 138 of mask which increases water surface area and encourages evaporation. The wicking is done by narrow groves on the inside of the mask.

Now referring to FIGS. 1-3, in this embodiment, a plurality of air channels are provided. FIGS. 1-3 illustrate an embodiment having a plurality of air channels 110, 114. In this embodiment, a two-part assembly is provided to aid in manufacturing and installing of copper mesh. Openings on the inside of the mask have adjusted sizes to account for pressure variances and provide equal flow through each channel. This delivers distributed flow inside of mask so user does not feel air pushing on face. The air channels 110, 114 may be slanted upward to direct condensed water into mask.

A copper mesh 131 is provided as a heat exchanger. This mesh could be in a multitude of channels. It should be understood that any material or other metal may be used if it meets the properties and characteristics desirable for hear exchange during breathing of the user. In the present embodiment, as disclosed, a copper mesh is utilized. In this embodiment, copper is desirable for its high thermal density and thermal conduction properties. Heat exchangers are devices that transfer heat in order to achieve desired heating or cooling. An important design aspect of heat exchanger technology is the selection of appropriate materials and geometry to conduct and transfer heat quickly and efficiently.

In the present embodiment, copper or other materials may be used to assist in warming the temperature of the air though the heat exchanger. In one embodiment, copper is used since copper has many desirable properties for thermally efficient and durable heat exchangers. First and foremost, copper is an excellent conductor of heat. This means that copper's high thermal conductivity allows heat to pass through it quickly. Other desirable properties of copper in heat exchangers include its corrosion resistance, biofouling resistance maximum allowable stress and internal pressure, creep rupture strength, fatigue strength, hardness, thermal expansion, specific heat , antimicrobial properties, tensile strength, yield strength, high melting point, alloyability, ease of fabrication, and ease of joining. The combination of these properties enable copper to be specified for heat exchangers in industrial facilities where heating, ventilation and air conditioning (HVAC) systems, vehicular coolers, radiators, and as heat sinks are used to cool computers, disk drives, televisions, computer monitors, and other electronic equipment. Copper is also incorporated into the bottoms of high-quality cookware because the metal conducts heat quickly and distributes it evenly. Non-copper heat exchangers are also available. Those skilled in the art will also recognize that alternative materials such as aluminum, carbon steel, stainless steel, nickel alloys, and titanium may also be used. Any of the above described materials or configurations may be used without undue experimentation in various embodiments described herein to achieve the desired results.

Additionally, the copper alloy is utilized not only for its high properties as a heat exchanger, but also because of copper's high antimicrobial properties. As a background, elevated copper levels inside a cell causes oxidative stress and the generation of hydrogen peroxide. Under these conditions, copper participates in a chemical reaction causing oxidative damage to microorganisms and/or pathogens. It has been shown that excess copper causes a decline in the membrane integrity of microbes, leading to leakage of specific essential cell nutrients, such as potassium and glutamate. This leads to desiccation and subsequent death of the cells forming the microorganism.

Furthermore, while copper is needed for many protein functions, in an excess situation (as on a copper alloy surface), copper binds to proteins that do not require copper for their function. This “inappropriate” binding leads to loss-of-function of the protein, and/or breakdown of the protein into nonfunctional portions. Accordingly, copper also increases the overall antimicrobial properties of the mask. In some embodiments, the copper mesh may be silver plated to achieve antimicrobial properties. Alternatively, any other coating or surface may be used which has antimicrobial properties. An antimicrobial surface or coating contains an antimicrobial agent that inhibits the ability of microorganisms to grow on the surface of a material. The most common and most important use of antimicrobial coatings has been in the healthcare setting for sterilization of medical devices to prevent hospital associated infections.

The present specification utilizes these properties as applied to the present face mask. In this embodiment, the silver prevents bacteria to grow when in contact with the human body which allows for the transmission of infectious disease. Antimicrobial surfaces are functionalized in a variety of different processes. A coating may be applied to a surface that has a chemical compound which is toxic to microorganisms. Other surfaces may be functionalized by attaching a polymer, or polypeptide to its surface. In this embodiment, the copper mesh is silver plated for antimicrobial properties. Furthermore, in this embodiment, the copper mesh 131 is cut to aid in assembly. Specifically, the copper mesh is cut of one or more of the same profiles rather than filling each channel with mesh. Corrugated cellulose paper or a heat moisture exchanger (HME) foam could be used to additionally increase the humidity regulation of the face mask. A phase change material such as calcium chloride can also be used to additionally increase the humidity and temperature regulation of the face mask.

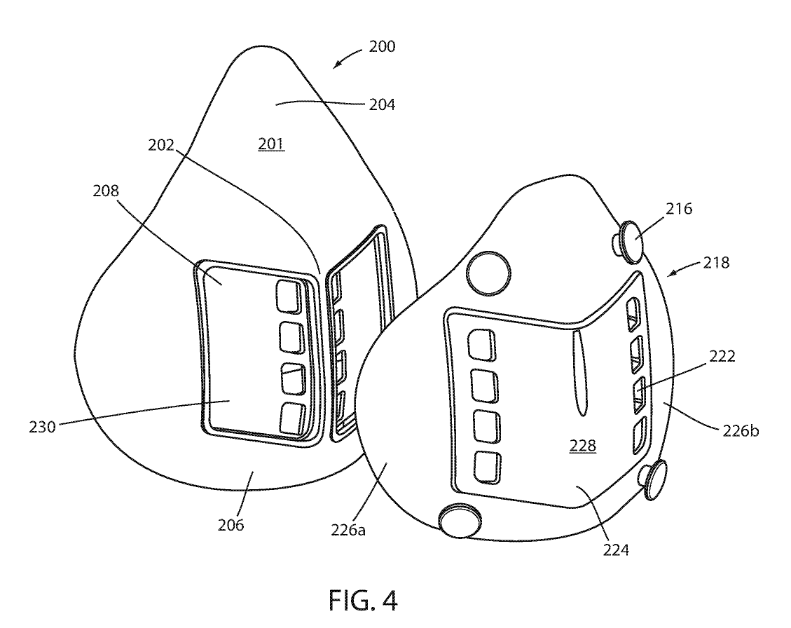

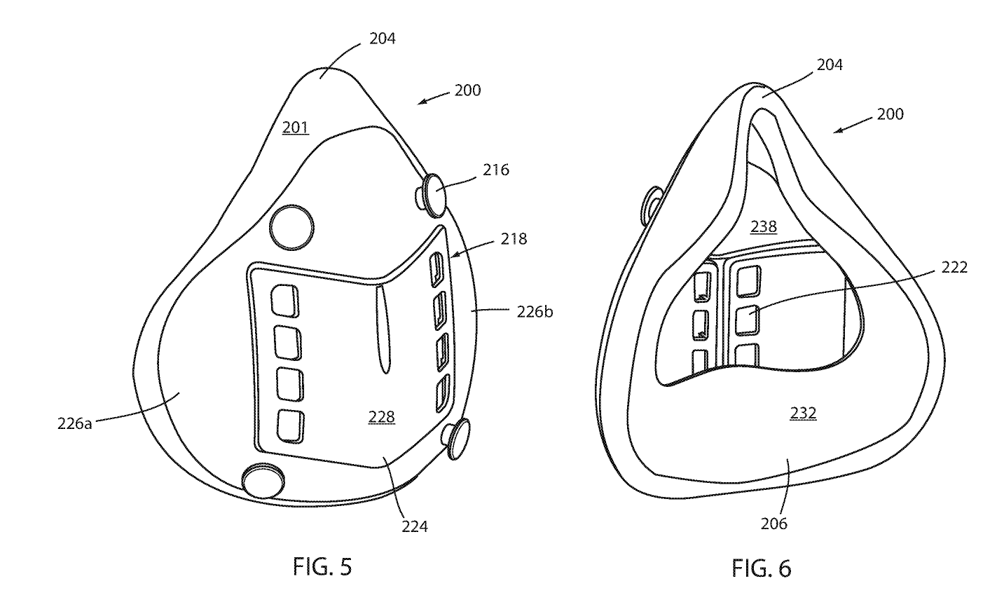

Referring now to FIGS. 4-6, in this alternative embodiment, a mask 200 is provided. The mask 200 generally includes a body 201 having a central portion 202. An upper portion 204 and a lower portion 206 are provided as reference points. The body 201 is preferably made of a silicone or other suitable material having properties similar to silicone, plastic or equivalent materials. The body 201 is shaped so as to confirm, or at least partially follow, the contour of the user's face. The upper portion is designed to fit over the user's nose while the lower portion rests at the user's chin, or just below the mouth (depending on the exact design). In use, the mask is configured to cover both the nose and the mouth to assist in temperature and humidity regulation of air exhaled and inhaled form both the nose and the mouth. Moreover, a textile garment (as illustrated in FIG. 9) is provided to connect to the grommet hooks 216. The textile garment is configured to wrap around the back of the user's head to hold the mask in place. The textile garment may also be provided to provided additional warmth to the user.

The mask 200 further includes a cover 218 configured to cover the air channels or passageways. The assembly of component 218 and 208 creates the channels in which air passes through. Within this channel copper mesh 230 could be placed so the user's breath comes in contact with it. The cover is configured to assist in the manufacturing and assembly process. The cover includes an outer surface 228 and side portions 226a and 226b and a bottom 224, incorporated for reference. The cover may include a plurality of apertures 222 to further assist and regulate airflow to the air channels on the body 201 of the mask 200.

The rear of the mask 200 includes a surface 232. The surface 232 is configured to also contour the shape of the user's face while the mask is in user. The mask 200 includes an inner surface 238 which may also include an antimicrobial surface coating (i.e. such as silver).

These methods of temperature and humidity regulation can be applied to other embodiments that may include the following: a) only regulating the air that passes through the user's nose; b) only regulating the air that passes through the user's mouth; or c) being installed into pre-existing or modular products. An example of this technology implementation is integrating it into a cartridge of similar design to the 3M filter cartridges. This would allow the user to wear a cartridge compliant mask and breathe warm comfortable air through the developed regulation technology.

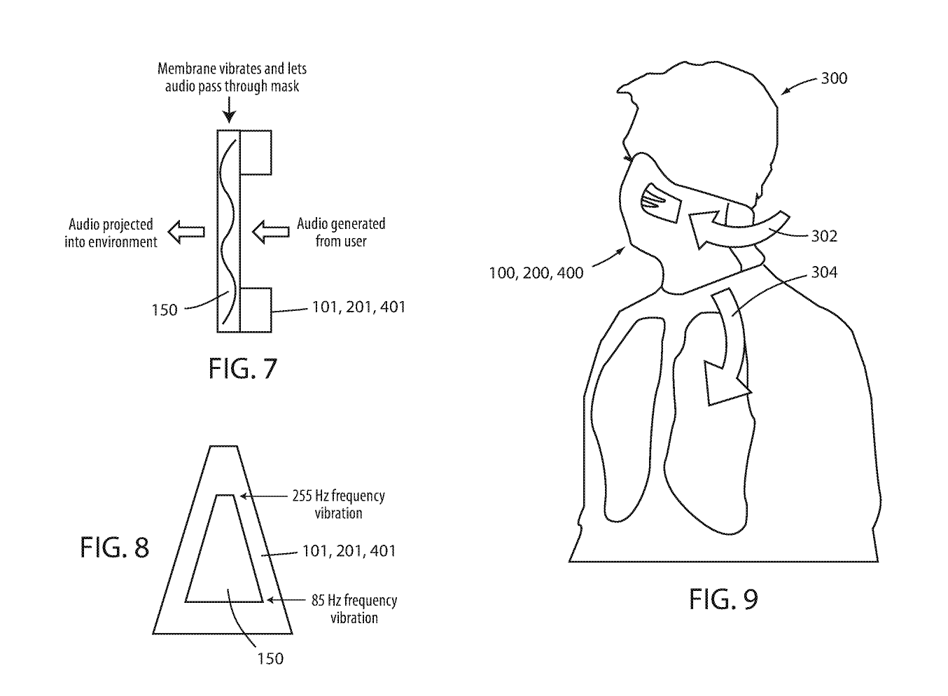

FIG. 7 and FIG. 8 illustrate a passive speaking membrane 150 that may be connected to or is part of the mask body 101, 201. The passive speaking membrane 150 is a thin membrane which passes sound vibrations through the mask. This membrane could have various positions one including in front of the mouth. In the embodiment as illustrated in FIG. 8, the passive speaking membrane 150 is tapered with a resonance frequency between 85 Hz and 255 Hz to allow the human voice frequency range to pass through and resonate. Those skilled in the art will recognize that various membrane geometries can be used including those having circles as a circular configuration has the most nodal resonance frequencies.

Referring now to FIG. 9, an embodiment is illustrated with the mask shown on a user. The top of the user 300 can be either exposed or insulated with textile garment. The textile garment can be used to hold the face mask to the user. Those skilled in the art will recognize that alternative forms can also be used to couple and/or hold the mask to the user including but not limited to elastic, neoprene, or string.

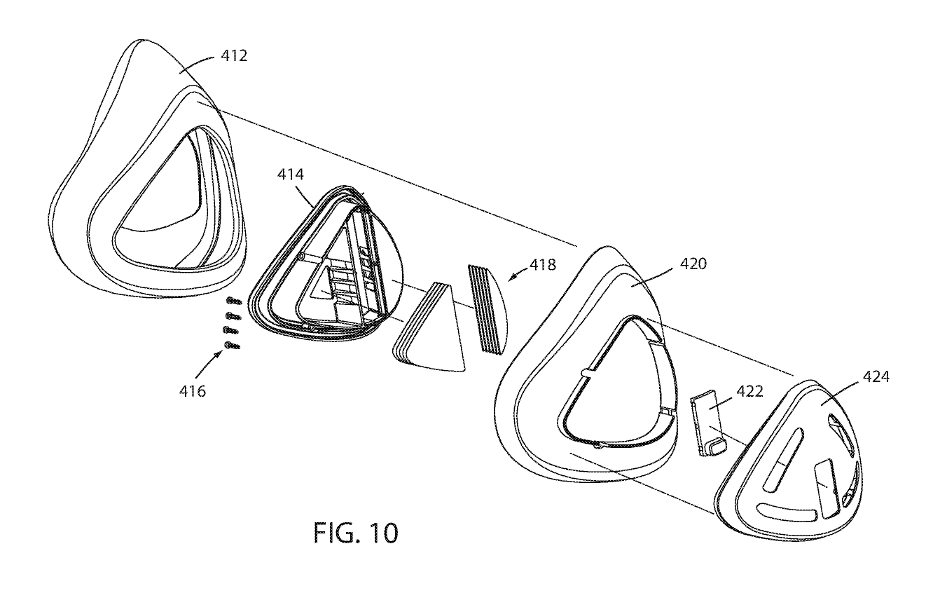

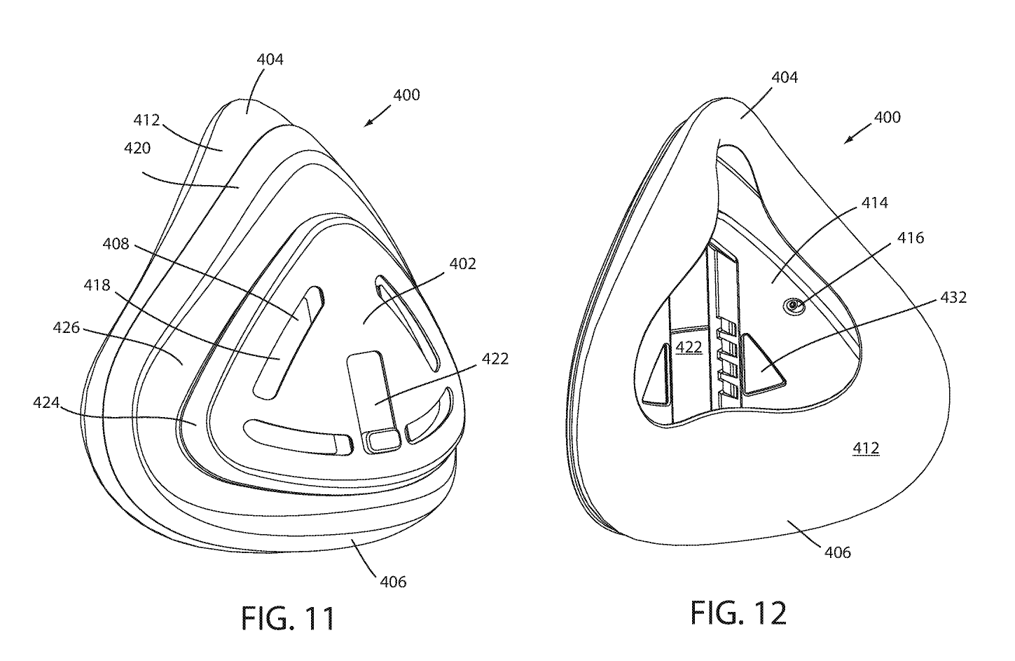

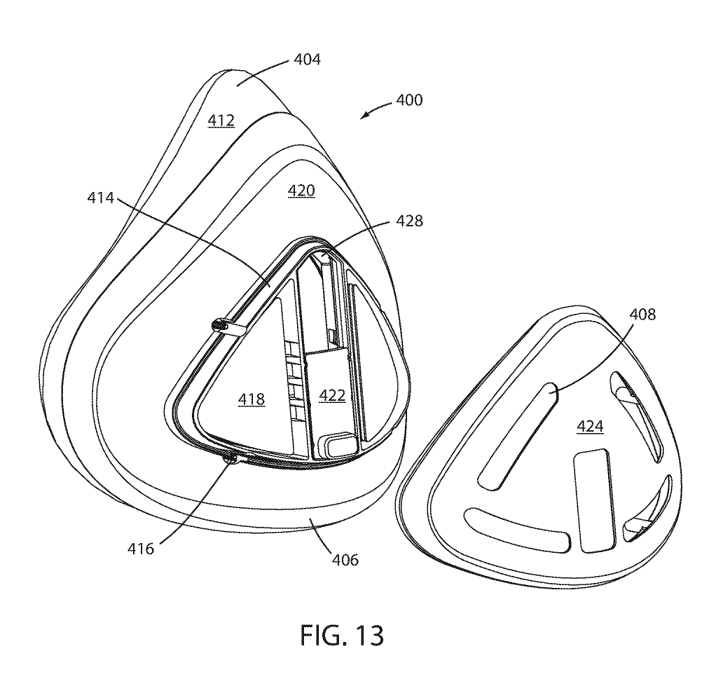

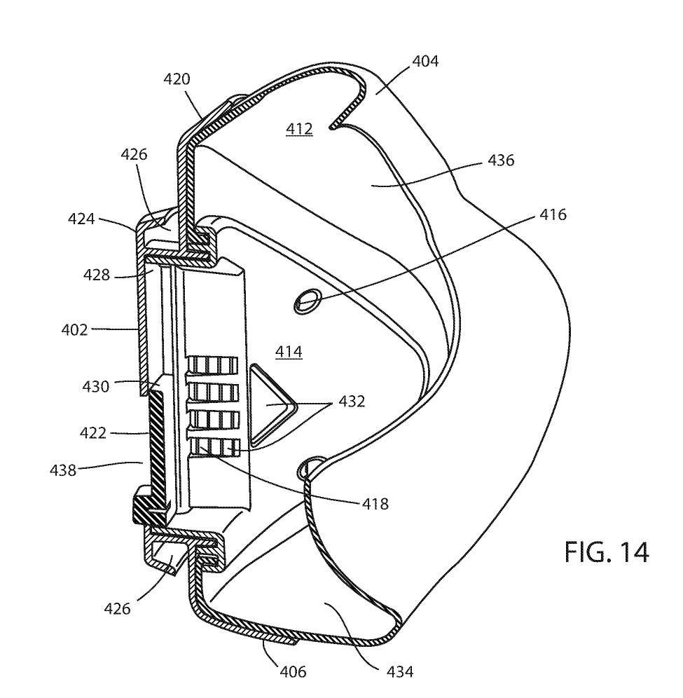

Referring now to FIGS. 10-14, in still yet another alternative embodiment of the invention, the mask 400 generally includes a body 412 having a central portion 402, an upper portion 404 and a lower portion 406 are provided as reference points. The body 412 is preferably made of a silicone or other suitable material having properties similar to silicone, plastic or equivalent materials. An alternative to silicone includes thermally enhanced plastic (TEP) which is a flexible and can be a biocompatible material. The body 412 is shaped cover the user's nose and mount and to confirm, or at least partially follow, the contour of the user's face. It is also shaped so it can couple to the other components to the mask 400. The upper portion is designed to fit over the user's nose. The fit between the upper portion of 412 and the user's nose varies and can cover a various proportion of the user's nose. The lower portion rests at the user's chin, under the user's chin, or just below the mouth (depending on the exact design and user face size). The mask is configured to cover both the nose and the mouth to assist in temperature and humidity regulation of air exhaled and inhaled form both the nose and the mouth. Layer 420 could be more rigid than layer 412 so it can be used as a support and prevent body 412 from deforming too much when pressed to the user's face. Furthermore, a textile garment (as illustrated in FIG. 9) is provided to connect to the annular groove 426. The textile garment is configured to wrap around the back of the user's head to hold the mask in place. The textile garment may also be provided to provided additional warmth to the user. The textile garment can also cover the top of the user's head.

The mask 200 further includes layers 414, 420, 422, and 424 to create multiple air channels or passageways. The assembly of these component creates the channels in which air passes through. A multitude of layers are needed to assist in the manufacturing and assembly process. Those skilled in the art will recognize that a fewer or greater number of layers could also be used. The heat exchanging material 418 is positioned between layers 414 and 422. Material 418 can be sheets of perforated aluminum stacked on top of each other in order to fill the channels and capture and release enough thermal energy.

Layer 414 may include a plurality of apertures 432 to further assist and regulate airflow to the air channels on the layer 412 of the mask 400. Body 424 may include a plurality of apertures 408 to further assist and regulate airflow to the air channels on the body 412 of the mask 400. A feature that this embodiment includes is a thermal override that could be manual or automatic. Slider 422 is able to move within bodies 424 and 414 vertically in line with the top and bottom of layer 412. The top of slider 422 could move from point 430 to point 428 and create a variable sized opening. This opening could be a singular of multitude of openings. When a user breaths' through the mask and the slider 422 is up, some of the air will pass through the opening and some will go through the heat regulating channels. This will reduce the humidity and temperature regulating efficiency and allow the user to breathe colder air.

When the user exhales, air passes through apertures 432 and contacts the material 418. At this stage in the air regulating process, the material 418 is colder than the user's breath. The humidity in the user's breath might condense into water and flow into or outside of the mask. If the water goes into the mask, it might first flow to surface 434. Surface 434 or 436 might have a texture that leverages the surface tension of water to flow some of the condensed water from surface 434 to surface 436. This might increase the surface area of the water. The environment within body 412 might be warm and the previously condensed water might evaporate and re-humidify the air when the user breathes in. The mask 400 includes inner surfaces 434 and 436 which may also include an antimicrobial surface coating (i.e. such as silver) for inhibiting the growth of pathogens and/or microorganisms.

The presented embodiment could use screws 416 to couple the components together. An alternative could be using plastic snaps or adhesion between different featuring bodies. It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter.

Moreover, although various aspects of the claimed subject matter have been described herein, such aspects need not be utilized in combination. It is therefore intended that the appended claims cover all such changes and modifications that are within the scope of the claimed subject matter.

We claim:

1. A facemask for controlling temperature and humidity of inhaled breath comprising:
a substantially curved body configured to cover a user's nose and mouth;
a central portion configured within the curved body;
a heat exchanger positioned on the inside of the central portion; and
wherein the central portion includes a plurality of apertures which air flows to the user.

2. A facemask as in claim 1, wherein the heat exchanger is a metallic permeable barrier positioned within each of the plurality of apertures.

3. A facemask as in claim 1, metallic permeable barrier is covered with an element or compound for deterring the growth of microorganisms.

4. A face mask as in claim 1, wherein the size of the plurality of apertures is adjustable for controlling the volume of air moving into and out of the mask.

5. A face mask as in claim 1, further comprising a speaking membrane configured within the body and tailored in frequency to allow a user's voice to be heard outside the mask.

6. A face mask as in claim 1, further comprising an annular groove to couple to the textile and enable the mask to be attached to the user's head.

7. A face mask as in claim 1, wherein the body is formed using a plurality of stacked layers.

8. A face mask for use in cold ambient temperatures comprising:
a body configured in a substantially curved shape for covering the user's nose and mouth where the body includes a first plurality of air chambers and a second plurality of air chambers on respective side of the body;
a heat exchanger positioned on the inside of the first plurality of air chambers and the second plurality of air chambers for capturing and releasing heat to the exchanged air moving into the first plurality of air chambers and second plurality of air chambers; and
wherein first plurality of air chambers and the second plurality of air chambers include a plurality of apertures that are sized and positioned to control the temperature, humidity and air flow of air inhaled and exhaled though the body.

9. A facemask as in claim 8, wherein heat exchanger is configured in the form of a metallic permeable barrier.

10. A facemask as in claim 9, metallic permeable barrier is covered with an element or compound for deterring the growth of microorganisms.

11. A face mask as in claim 8, wherein the size of the plurality of apertures is adjustable for controlling the volume of air moving into and out of the mask.

12. A face mask as in claim 8, wherein the plurality of apertures are substantially rectangular shaped.

13. A face mask as in claim 8, further comprising a speaking membrane configured within the body and tailored in frequency to allow a user's voice to be heard outside the mask.

14. A face mask as in claim 8, further comprising an annular groove to couple to the textile and enabling the mask to be attached to the user's head.

15. A face mask for regulating heat and humidity of the user's inhaled breath in cold temperatures comprising:
a body configured for covering the nose and mouth of the user's face where the body has a plurality of air channels therein;
a cover having a plurality of apertures therein for partially covering the air channels; and
a metallic permeable barrier configured on an inside of the plurality of channels and operating as a heat exchanger.

16. A facemask as in claim 15, where the plurality of apertures are configured into two groups on opposite sides of a center of the mask.

17. A facemask as in claim 15, wherein the plurality of apertures are in a substantially rectangular shape.

18. A face mask as in claim 15, further comprising a speaking membrane configured within the body and tailored in frequency to allow a user's voice to be heard outside the mask.

19. A face mask as in claim 15, further comprising an annular groove to couple to the textile and enabling the mask to be attached to the user's head.

20. A facemask as in claim 15, metallic permeable barrier is covered with an element or compound for deterring the growth of microorganisms.
